(19) United States
(12) Patent Application Publication
(10) Pub. No.: US 2021/0085422 A1
(43) Pub. Date: Mar. 25, 2021

(54) Breast Positioning Device for Breast Cancer Radiation Therapy

(71) Applicants: Suzanne Rossel, Lachine (CA); Nancy Raffis, Laval (CA); Diane Rousseau, Candiac (CA)

(72) Inventors: Suzanne Rossel, Lachine (CA); Nancy Raffis, Laval (CA); Diane Rousseau, Candiac (CA)

(21) Appl. No.: 17/027,128

(22) Filed: Sep. 21, 2020

Related U.S. Application Data
(60) Provisional application No. 62/905,601, filed on Sep. 25, 2019.

(51) Int. Cl. A61B 90/17; A61N 5/10

(52) U.S. Cl. CPC A61B 90/17; A61N 2005/1097; A61N 5/10

(57) ABSTRACT

A breast positioning device for breast cancer radiation therapy includes i) a body portion made of a stretchable synthetic fabric and having a generally tubular shape configured for tight fitting on a person torso, ii) a bra cup on a first portion on the body portion, defining a first lateral side thereof, and iii) a flat portion on a second portion on the body portion, defining a second lateral side, the second portion being positioned laterally symmetrical to the first portion on the body portion. The bra cup is thermoformed in the stretchable synthetic fabric to have a greater transparency than surroundings thereof rendering a first breast therein visible while immobilizing the first breast therein in a predetermined position. When the breast positioning device is worn by a person during the breast cancer radiation therapy, the bra cup immobilizes the first breast therein in the predetermined position which is both visible and suitable for treatment while forcing a second breast in a flattened position away from the first lateral side.

### CROSS REFERENCE TO RELATED PATENT APPLICATION

This application claims the benefit of U.S. Provisional Application No. 62/905,601, filed on Sep. 25, 2019, the content of which is incorporated by reference herein.

### FIELD

The present disclosure generally relates to radiation therapy. More specifically, the present disclosure concerns a breast positioning device for breast cancer radiation therapy.

### BACKGROUND

It is estimated that there are more than 1,000,000 new cases of breast cancer every year worldwide. Most women with breast cancer will have surgery, radiation therapy being almost always given after breast-conserving surgery.

It is well-known that a more precise radiation therapy leads to less unwanted irradiation to the internal organs and surrounding tissues. Precise breast positioning is therefore key to the quality of the radiation treatment (RT).

Irradiation of the breast is a technically complex procedure. To ensure accurate delivery of the RT dose, patients must be treated in a position that can be reproduced at each treatment fraction.

Patient positioning is most effective when it renders the patient and breast stable during treatment and is easy to reproduce.

Devices are known that are shaped to be positioned on a patient's upper body and which comprise means for positioning one of the breasts for a radiotherapy beam while trying to position the other breast out of the radiotherapy treatment field.

An example of such a device is described in International Patent Application Publication No. WO2015/013806, published on Feb. 5, 2015, naming Raffis et al as inventors, and titled “Breast Immobilization Device for Radiotherapy”. This device is in the form of full upper body camisole, which includes first and second breast holding cups, and a plurality of hooks and straps, including on the back and on the sleeves, to allow full adjustment of the garment.

One drawback of this device by Raffis et al. is that the breast that is not to be treated is raised by a breast holding cup, and as such may remain vulnerable to radiations. It is also relatively bulky and uncomfortable. Furthermore, even though it includes many hooks and straps, it lacks versatility considering that a different version is to be made for therapy for the right or left breast. It has also been found difficult to adjust.

United States Publication No. 2012/0220811, published on Aug. 30, 2012, naming Thomson as the inventor, and titled “Radiation Treatment Method for Use with a Radiation Treatment Bra”, describes a radiation treatment bra including a plurality of adjustable elements. While the bra is relatively simpler and lighter, and therefore subjectively more comfortable than the device by Raffis, it shares therewith the drawbacks of the noted lack of adjustability and of versatility and the fact that the untreated breast is raised by a breast holding cup.

A breast positioning device for breast cancer radiation therapy that is free of the above-described drawbacks is therefore desirable.

### SUMMARY

The lack of protection for the untreated breast in a breast cancer radiation therapy is solved by forcing the untreated breast in a flattened position while bringing the other breast in a suitable immobilized and visible position for treatment.

The resulting device includes a single bra cup and therefore can be made reversible so as to be suitable for breast cancer treatment for any one of the right and left breasts.

According to an illustrative embodiment, there is provided a breast positioning device for breast cancer radiation therapy comprising:

a body portion made of a stretchable synthetic fabric and having a generally tubular shape configured for tight fitting on a person torso;

a bra cup on a first portion on the body portion, defining a first lateral side thereof; the bra cup being thermoformed in the stretchable synthetic fabric so as to immobilize a first breast in a predetermined position therein and to yield a greater transparency for the first bra cup than surroundings thereof, rendering the first breast therein visible; and

a flat portion on a second portion on the body portion, defining a second lateral side thereof; the second portion being positioned laterally symmetrical to the first portion on the body portion;

whereby, when the breast positioning device is worn by a person during the breast cancer radiation therapy, the bra cup immobilizes the first breast on the first lateral side in the predetermined position therein, which is both visible and suitable for treatment while forcing a second breast in a flattened position away from the first lateral side.

According to another illustrative embodiment, there is provided a breast positioning device for breast cancer radiation therapy comprising:

a body portion made of a stretchable synthetic fabric and having a generally tubular shape configured for tight fitting on a person torso; the body portion being in the form of a generally rectangular piece of the stretchable synthetic fabric, which includes two opposite lateral edges that are joinable to define the generally tubular shape;

an extension band that is attachable to both lateral edges therebetween for their joining;

two shoulder straps attached to the body portion and defining first and second lateral sides of the body portion; and

a bra cup on the first lateral side of the body portion; the bra cup being thermoformed in the stretchable synthetic fabric so as to immobilize a first breast in a predetermined position therein and have a greater transparency than surroundings thereof rendering the first breast therein visible; a portion on the second lateral side, which is laterally symmetrical to a position of the bra cup on the first lateral side, being flat;

whereby, when the breast positioning device is worn by a person during the breast cancer radiation therapy, the bra cup immobilizes the first breast on the first lateral side in the predetermined position therein, which is both visible and suitable for treatment while forcing a second breast in a flattened position away from the first lateral side.

According to still another illustrative embodiment, there is provided a garment for breast cancer radiation treatment comprising a tubular stretchable body having a single bra cup that is thermoformed on a first lateral side thereof and that has a greater transparency than a remaining of the tubular stretchable body, and a flat portion that is symmetrically opposite the bra cup on a second lateral side thereof.

Such breast positioning devices allows placing a breast in a same position relatively to the patient morphology, and this for each treatment.

Other objects, advantages, and features of the breast positioning device for breast cancer radiation therapy will become more apparent upon reading the following non-restrictive description of preferred embodiments thereof, given by way of example only with reference to the accompanying drawings.

### BRIEF DESCRIPTION OF THE DRAWINGS

In the appended drawings:

FIG. 1 is a front perspective of a breast positioning device for breast cancer radiation therapy according to a first illustrative embodiment; and

FIG. 2 is a top plan view of the device from FIG. 1, the device being illustrated as worn by a person.

### DETAILED DESCRIPTION

In the following description, similar features in the drawings have been given similar reference numerals, and in order not to weigh down the figures, some elements are not referred to in some figures if they were already identified in a precedent figure. Herein, it shall further be noted that, for avoiding unnecessary details obscuring the invention, only device structures and/or processing steps closely relevant to schemes according to the invention are shown in the accompanying drawings while omitting other details less relevant to the invention.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, un-recited elements.

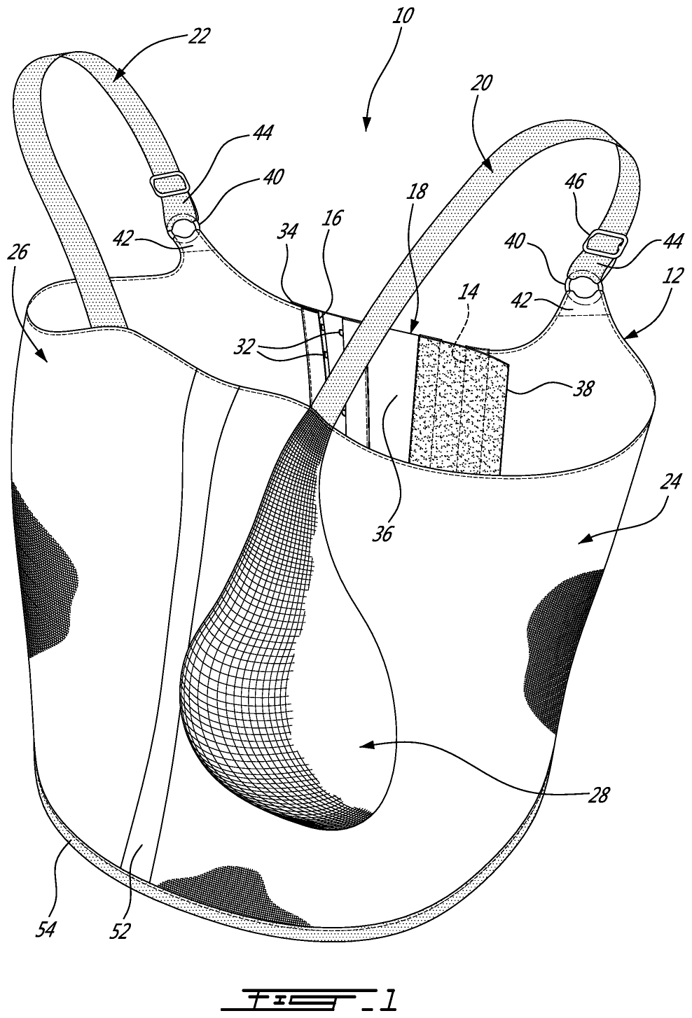

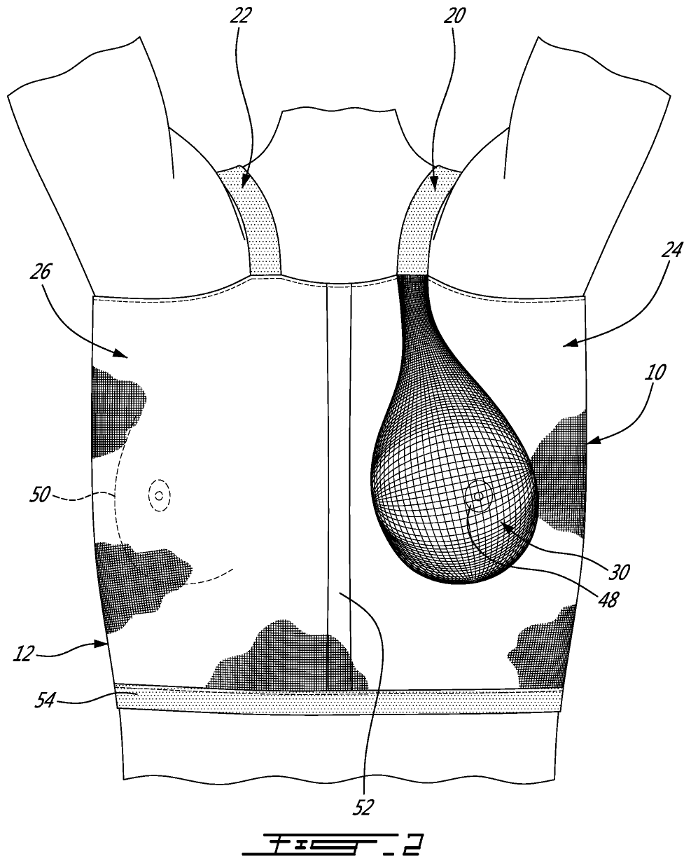

An illustrative embodiment of a breast positioning device 10 for breast cancer radiation therapy will now be described with reference first to FIGS. 1 and 2.

The breast positioning device 10 comprises a body portion 12 having two lateral edges 14-16, an extension band 18 that is attachable to both lateral edges 14-16 for their joining, yielding a generally tubular shape for the body portion 12, two shoulder straps 20-22 attached to the body portion 12 and defining first and second lateral sides 24-26 of the body portion 12, and a bra cup 28 formed in the body portion 12 on the first lateral side 24 thereof.

Each of these parts of the device 10 will now be described in more detail.

The body portion 12 is made of a stretchable synthetic fabric, such as, for example, polyester and/or nylon. As will be described hereinbelow in more detail, the breast positioning device 10 includes features, in addition to its material, that contribute to tight fitting the body portion 12 on a person torso and maintain in position a first breast 30 during radiation therapy.

The extension band 18 is provided to allow a one-size body portion 12 to fit a plurality of torso sizes. The extension band 18 includes first series of parallel rows of eyes 32 near a first edge 34 on a first face 36 thereof and a second series of parallel rows of eyes (not shown) near a second edge 38 on the opposite face thereof (not shown).

Respective rows of complementary hooks (not shown) are provided on both lateral edges 14 and 16 of the body portions 12 for selective attachment to the extension band 18.

A person skilled in the art will appreciate that: 1) the first series of eyes' rows yields a plurality of attaching positions for the body portion 12, adapted for a corresponding plurality of torso sizes; and 2) providing attachments on both side of the extension band 18 allows facilitating the attachment of the main body 12 on the illustrated side, but also when it is reversed inside out so that the bra cup 28 finds itself on the opposite lateral side of the device 10. It yields from that last feature, that a same device 10 can be used for breast cancer radiation therapy of any one of the two breasts 30 and 50.

The main part of the extension band 18 can be made of the same fabric as the body portion 12 or of another material.

The shoulder straps 20-22 are attached to the body portion 12 using stiches on the front and a ring 40 on the back. Each ring 40 is secured to both a loop portion 42, 44 in the body portion 12 and a respective strap 20, 22. Other attachment mechanisms can be used to the secure the straps 20-22 on the front and back of the body portion 12.

A buckle 46 is further provided on each strap 20 and 22, which allows conventionally adjusting the length thereof. According to another embodiment (not shown), such an adjustment mechanism is omitted on the straps 20-22, wherein their lengths are tailored for a specific physiognomy.

The bra cup 28 is thermoformed in the synthetic fabric of the body portion 12 using a corresponding heated shape on one side and a complementary shape on the other side (both not shown). Such a process allows stretching the synthetic fiber, resulting in a transparency that allows visual repairing of a breast 30 and more specifically of its nipple 48. Moreover, the thermoforming greatly reduces the stretchability of the fabric after the process, resulting in a cup 28 having a predetermined and fixed configuration. Such a feature of the cup, and its transparency, have been found to improve the daily reproducibility of the treatment.

Moreover, the bra cup 28 being pre-formed within the body portion 12 of the device 10 contributes to raising and firmly maintaining in place the breast 30 during treatment.

While the bra cup 28 according to the illustrated embodiment has been described as formed within the body portion 12, it can alternatively be thermoformed in a separate piece of fabric (not shown) which is then attached to the main body 12, using for example stiches, an adhesive or else).

The portion on the second lateral side 26, which is laterally symmetrical to a position of the bra cup 28 on the first lateral side, is free of a cup, which allows flattening the other breast away from the cup 28 during treatment. This contributes to the protecting the un-treated breast 50 from radiation during treatment.

As a person skilled in the art will now appreciate, the device 10, and more specifically the cup 28 can be adapted to accommodate a specific breast size.

The body portion 12 further includes a strip 52 of fabric that extends along the height of the body portion 12 in the center thereof equidistantly from both straps 20 and 22. The strip 52 can be used as a reference positioning mark during treatment. Considering that the device 10 is reversable, the strip 52 extends on both sides of the body portion to also be visible when the device 10 is reversed inside out.

According another embodiment, the strip 52 is provided only on one side of the device 10. According to still another embodiment, the strip 52 is in the form of markings (not shown) on the body portion 12. The strip 12 or any markings can also be omitted.

The body portion 12 further includes an underband 54 at the bottom edge thereof. According to another embodiment (not shown), the underband 54 is omitted.

It is to be noted that many modifications could be made to the device 10 described hereinabove and illustrated in the appended drawings. For example:
the straps can be omitted;
other attachments than the illustrated eyes and hooks, such as Velcro™, snap buttons, or the likes can be used to attach the body portion 12;
the extension band 18 can be omitted, with a single or a plurality of attachment rows be provided directly on the body portion 12;
the attachments on the two lateral edges 14-16 of the body portion 12 can be replaced by a seam or another similar non-detachable elements, yielding a slip-on body portion;
the configuration and size of the body portion 12 may be different than illustrated.

Although a breast positioning device 10 for breast cancer radiation therapy has been described hereinabove by way of illustrated embodiments thereof, it can be modified. It is therefore to be understood that numerous modifications may be made to the illustrative embodiments and that the scope of the claims should not be limited by the preferred embodiment, but should be given the broadest interpretation consistent with the description as a whole.

What is claimed is:

1. A breast positioning device for breast cancer radiation therapy comprising:
a body portion made of a stretchable synthetic fabric and having a generally tubular shape configured for tight fitting on a person torso;
a bra cup on a first portion on the body portion, defining a first lateral side thereof; the bra cup being thermoformed in the stretchable synthetic fabric so as to immobilize a first breast in a predetermined position therein and to yield a greater transparency for the first bra cup than surroundings thereof, rendering the first breast therein visible; and
a flat portion on a second portion on the body portion, defining a second lateral side thereof; the second portion being positioned laterally symmetrical to the first portion on the body portion;
whereby, when the breast positioning device is worn by a person during the breast cancer radiation therapy, the bra cup positions the first breast on the first lateral side in the predetermined position therein, which is both visible and suitable for treatment while forcing a second breast in a flattened position away from the first lateral side.

2. The device of claim 1, wherein the body portion is in the form of a generally rectangular piece of the stretchable synthetic fabric, which includes two opposite lateral edges that are joinable to define the generally tubular shape.

3. The device of claim 2, wherein the two opposite lateral edges of the body portion include complementary attachments for joining the two opposite lateral edges.

4. The device of claim 3, further comprising an extension band that is attachable to both lateral edges of the body portion therebetween for their joining.

5. The device of claim 4, wherein the extension band includes two lateral edges, each including complementary attachments for cooperation with attachments on a respective lateral edges of the body portion.

6. The device of claim 5, wherein the complementary attachments are positioned in at least two rows, each defining an attaching position that is adapted to one of a plurality of torso sizes.

7. The device of claim 5, wherein the complementary attachments on each of the lateral edges of the extension band are mounted on an opposite side face of the extension band;
whereby, at least one of the complementary attachments on each of the lateral edges of the extension band are accessible from outside of the generally tubular shape when the body portion is revered inside out.

8. The device of claim 2, wherein the first and second portions of the body portion define a front side thereof and the two opposite lateral edges define a rear side thereof when they are joined.

9. The device of claim 8, further comprising a strip of fabric attached to the body portion so as to extend generally along the height thereof equidistantly from the two opposite lateral edges of the body portion; the strip of fabric defining a reference positioning mark.

10. The device of claim 9, wherein the strip of fabric extends on both the front and rear sides of the body portion.

11. The device of claim 1, further comprising two shoulder straps attached to the body portion, each on a respective one of the first and second lateral sides.

12. The device of claim 11, wherein the length of each one of the two shoulder straps is adjustable.

13. The device of claim 1, wherein the bra cup is thermoformed.

14. The device of claim 1, wherein the synthetic fabric includes at least one of polyester and nylon.

15. The device of claim 1, wherein the body portion includes a bottom edge and further comprises an underband at the bottom edge.

16. A breast positioning device for breast cancer radiation therapy comprising:
a body portion made of a stretchable synthetic fabric and having a generally tubular shape configured for tight fitting on a person torso; the body portion being in the form of a generally rectangular piece of the stretchable synthetic fabric, which includes two opposite lateral edges that are joinable to define the generally tubular shape;
an extension band that is attachable to both lateral edges therebetween for their joining;
two shoulder straps attached to the body portion and defining first and second lateral sides of the body portion; and
a bra cup on the first lateral side of the body portion; the bra cup being thermoformed in the stretchable synthetic fabric so as to immobilize a first breast in a predetermined position therein and have a greater transparency than surroundings thereof rendering the first breast therein visible; a portion on the second lateral side, which is laterally symmetrical to a position of the bra cup on the first lateral side, being flat;
whereby, when the breast positioning device is worn by a person during the breast cancer radiation therapy, the bra cup immobilizes the first breast on the first lateral side in the predetermined position therein, which is both visible and suitable for treatment while forcing a second breast in a flattened position away from the first lateral side.

17. A garment for breast cancer radiation therapy comprising a tubular stretchable body having i) a single bra cup that is thermoformed on a first lateral side thereof and that has a greater transparency than a remaining of the tubular stretchable body, and ii) a flat portion that is symmetrically opposite the bra cup on a second lateral side thereof.
